SEQUENCE LISTING (29 sequences)

SEQ ID NO: 1 - DNA, 19 bp, Artificial Sequence
  Synthetic oligonucleotide
gacctctcattctgaaacc

SEQ ID NO: 2 - DNA, 19 bp, Artificial Sequence
  Synthetic oligonucleotide
gatctgaatcctggactcc

SEQ ID NO: 3 - DNA, 19 bp, Artificial Sequence
  Synthetic oligonucleotide
ccaggagctcatccagcag

SEQ ID NO: 4 - DNA, 19 bp, Artificial Sequence
  Synthetic oligonucleotide
cctaaagctcctccagcag

SEQ ID NO: 5 - DNA, 19 bp, Artificial Sequence
  Synthetic oligonucleotide
gtcctaaagctcctccagc

SEQ ID NO: 6 - DNA, 19 bp, Artificial Sequence
  Synthetic oligonucleotide
cccaggaaagaaaggcagt

SEQ ID NO: 7 - DNA, 19 bp, Artificial Sequence
  Synthetic oligonucleotide
gatgcccaggaaagaaagg

SEQ ID NO: 8 - DNA, 19 bp, Artificial Sequence
  Synthetic oligonucleotide
aacaagggcacagagaggc

SEQ ID NO: 9 - DNA, 19 bp, Artificial Sequence
  Synthetic oligonucleotide
atgtaactctaatccaggt

SEQ ID NO: 10 - DNA, 19 bp, Artificial Sequence
  Synthetic oligonucleotide
ctgaactaatcatccagga

SEQ ID NO: 11 - DNA, 19 bp, Artificial Sequence
  Synthetic oligonucleotide
actgtactgaatcataagg

SEQ ID NO: 12 - DNA, 1709 bp, Artificial Sequence
  Synthetic oligonucleotide
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctata

SEQ ID NO: 13 - DNA, 1574 bp, Artificial Sequence
  Synthetic oligonucleotide
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 14 - DNA, 19 bp, Artificial Sequence
  Synthetic oligonucleotide
ggtccaaacgagtctccgt

SEQ ID NO: 15 - DNA, 19 bp, Artificial Sequence
  Synthetic oligonucleotide
cgttctctggtggcgatgc

SEQ ID NO: 16 - DNA, 19 bp, Artificial Sequence
  Synthetic oligonucleotide
ggcgtgacctctcattctg

SEQ ID NO: 17 - DNA, 19 bp, Artificial Sequence
  Synthetic oligonucleotide
cctcgctgcctggctcacg

SEQ ID NO: 18 - DNA, 19 bp, Artificial Sequence
  Synthetic oligonucleotide
ccgtttctaggagaggttg

SEQ ID NO: 19 - DNA, 19 bp, Artificial Sequence
  Synthetic oligonucleotide
ccggtattcttcctcgctg

SEQ ID NO: 20 - DNA, 19 bp, Artificial Sequence
  Synthetic oligonucleotide
gagctcccttgcacgtcag

SEQ ID NO: 21 - DNA, 19 bp, Artificial Sequence
  Synthetic oligonucleotide
agccagaatttcacggaag

SEQ ID NO: 22 - DNA, 19 bp, Artificial Sequence
  Synthetic oligonucleotide
ttgcctagacagcgtcgga

SEQ ID NO: 23 - DNA, 19 bp, Artificial Sequence
  Synthetic oligonucleotide
tccaggagatgtaactcta

SEQ ID NO: 24 - DNA, 19 bp, Artificial Sequence
  Synthetic oligonucleotide
ctctgaactaatcatccag

SEQ ID NO: 25 - DNA, 19 bp, Artificial Sequence
  Synthetic oligonucleotide
tctctgaactaatcatcca

SEQ ID NO: 26 - DNA, 19 bp, Artificial Sequence
  Synthetic oligonucleotide
tatctctgaactaatcatc

SEQ ID NO: 27 - DNA, 19 bp, Artificial Sequence
  Synthetic oligonucleotide
tatatctctgaactaatca

SEQ ID NO: 28 - DNA, 19 bp, Artificial Sequence
  Synthetic oligonucleotide
tttaatatatctctgaact

SEQ ID NO: 29 - DNA, 19 bp, Artificial Sequence
  Synthetic oligonucleotide
ggcattttaatatatctct

(19) United States
(12) Patent Application Publication
(10) Pub. No.: US 2017/0009230 A1
(43) Pub. Date: Jan. 12, 2017

(54) COMPOSITIONS FOR INHIBITING DUX4 GENE EXPRESSION AND USES THEREOF

(71) Applicant: Idera Pharmaceuticals, Inc., Cambridge, MA (US)

(72) Inventors: Sudhir Agrawal, Shrewsbury, MA (US); Lakshmi Bhagat, Framingham, MA (US); Fu-Gang Zhu, Bedford, MA (US)

(21) Appl. No.: 15/204,114

(22) Filed: Jul. 7, 2016

Related U.S. Application Data
(60) Provisional application No. 62/190,021, filed on Jul. 8, 2015.

(51) Int. Cl. C12N 15/113; A61K 45/06; A61K 31/713

(52) U.S. Cl. CPC C12N 15/113; A61K 31/713; A61K 45/06; C12N 2310/11; C12N 2320/31

(57) ABSTRACT

The present invention is directed to compounds, compositions, and methods useful for modulating DUX4 mRNA or protein expression using gene silencing compounds comprising two or more single stranded antisense oligonucleotides that are linked through their 5′-ends to allow the presence of two or more accessible 3′-ends.

### RELATED APPLICATION

This application claims the benefit of U.S. Provisional Application No. 62/190,021, filed on Jul. 8, 2015. The entire teachings of the above application are incorporated herein by reference.

### BACKGROUND OF THE INVENTION

Field of the Invention

The present invention relates to compounds, compositions, and methods of use for the inhibition Double Homeobox 4 (DUX4) gene expression or for diagnosing, treating and/or preventing diseases and/or conditions that respond to the inhibition of DUX4 gene expression.

Summary of the Related Art

Facioscapulohumeral muscular dystrophy (FSHD) is an inherited form of muscular dystrophy and is one of the most common muscular dystrophies, arising generally in young adulthood and causing progressive wasting of facial and upper body muscles, with progressive skeletal muscle weakness generally developing in other areas of the body as well. Approximately 20% of individuals with this disorder will become wheelchair dependent.

The underlying pathogensis of the disease is complex and is still being fully elucidated, but recent studies have shown that overexpression of the myopathic Double Homeobox 4 (DUX4) gene, silent in normal muscle, plays a role in FSHD. The DUX4 gene is located within a D4Z4 repeat array on Chromosome 4, and encodes a transcription factor that activates paired-like homeodomain transcription factor 1 (PITX1). There are two transcript forms of hDUX4, variant 1 (NM_001306068.1, 1710 bp) and variant 2 (NM_001293798.1, 1574 bp). There is no murine homolog to human DUX4.

Early studies in murine models showed that modulation of DUX4 gene expression corrected DUX4-associated myopathy in mouse muscle. Thus, inhibition of DUX4 expression represents a promising area for development of a treatment for FSHD. Presently there is no treatment for FSHD.

Thus, there exists a need for treatments for myopathic diseases or disorders, including muscular dystrophies such as FSHD, and for diseases or disorders that would benefit from the reduced expression of a transcriptional activator of PITX1, or from modulation of PITX1.

### BRIEF SUMMARY OF THE INVENTION

The present invention is directed to compounds, compositions, and methods useful for modulating DUX4 mRNA or protein expression using gene silencing compounds comprising two or more single stranded antisense oligonucleotides that are linked through their 5′-ends to allow the presence of two or more accessible 3′-ends. The gene silencing compounds according to the invention effectively inhibit or decrease DUX4 mRNA or protein expression.

Provided herein are methods, compounds, and compositions for modulating expression of DUX4 mRNA and protein. In certain embodiments, compounds useful for modulating expression of DUX4 mRNA and protein are gene silencing compounds.

In certain embodiments, modulation can occur in a cell or tissue. In certain embodiments, the cell or tissue is in an animal. In certain embodiments, the animal is a human. In certain embodiments, DUX4 mRNA levels are reduced. In certain embodiments, DUX4 protein levels are reduced. Such reduction can occur in a time-dependent manner or in a dose-dependent manner.

Also provided are methods, compounds, and compositions useful for preventing, treating, and ameliorating diseases, disorders, and conditions. In certain embodiments, such diseases, disorders, and conditions are myopathic diseases, disorders, and conditions. In certain embodiments such myopathic diseases, disorders, and conditions include muscular dystrophies, including FSHD.

In certain embodiments, methods of treatment include administering a DUX4 mRNA or protein expression gene silencing compound or composition to an individual in need thereof.

### BRIEF DESCRIPTION OF THE FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts DUX4 mRNA, including target sites for exemplary gene silencing compounds of the invention.

FIG. 2 shows the results of primary screening in vector-based assays using exemplary gene silencing compounds of the invention. Compound names are based on their target sites of SEQ ID NO: 13 (see Tables 3 and 4).

FIG. 3A through FIG. 3C shows dose response curves in vector-based assays with gene silencing compounds of the invention.

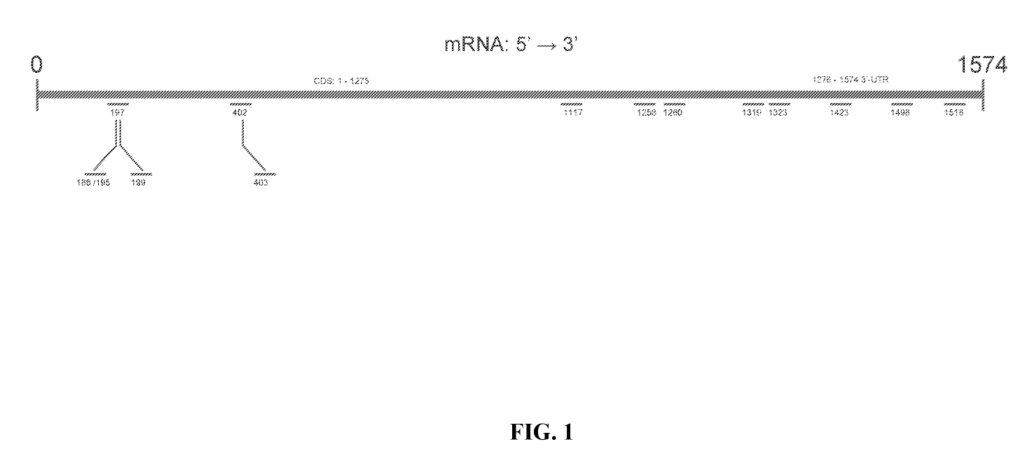

### DETAILED DESCRIPTION OF THE PREFERRED EMBODIMENTS

The invention relates to the therapeutic and prophylactic use of gene silencing compounds to down-regulate DUX4 mRNA or protein expression. Such molecules are useful, for example, in providing compositions for modulation of DUX4 gene expression or for treating and/or preventing diseases and/or conditions that are capable of responding to modulation of DUX4 gene expression in patients, subjects, animals or organisms.

The objects of the present invention, the various features thereof, as well as the invention itself may be more fully understood from the following description, when read together with the accompanying drawings in which the following terms have the ascribed meaning. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

The term “2′-O-substituted” means substitution of the 2′ position of the pentose moiety with an —O-lower alkyl group containing 1-6 saturated or unsaturated carbon atoms (for example, but not limited to, 2′-O-methyl), or with an —O-aryl or allyl group having 2-6 carbon atoms, wherein such alkyl, aryl or allyl group may be unsubstituted or may be substituted, (for example, with 2′-O-methoxyethyl, ethoxy, methoxy, halo, hydroxyl, trifluoromethyl, cyano, nitro, acyl, acyloxy, alkoxy, carboxyl, carbalkoxyl, or amino groups); or with a hydroxyl, an amino or a halo group, but not with a 2′-H group. In some embodiments the oligonucleotides of the invention include four or five 2′-O-alky nucleotides at their 5′ terminus, and/or four or five 2′-O-alky nucleotides at their 3′ terminus.

The term “3”, when used directionally, generally refers to a region or position in a polynucleotide or oligonucleotide 3′ (toward the 3′end of the nucleotide) from another region or position in the same polynucleotide or oligonucleotide.

The term “3′ end” generally refers to the 3′ terminal nucleotide of the component oligonucleotides. “Two or more oligonucleotides linked at their 3′ ends” generally refers to a linkage between the 3′ terminal nucleotides of the oligonucleotides which may be directly via 5′, 3′ or 2′ hydroxyl groups, or indirectly, via a non-nucleotide linker. Such linkages may also be via a nucleoside, utilizing both 2′ and 3′ hydroxyl positions of the nucleoside. Such linkages may also utilize a functionalized sugar or nucleobase of a 3′terminal nucleotide.

The term “5”, when used directionally, generally refers to a region or position in a polynucleotide or oligonucleotide 5′ (toward the 5′end of the nucleotide) from another region or position in the same polynucleotide or oligonucleotide.

The term “5′ end” generally refers to the 5′ terminal nucleotide of the component oligonucleotides. “Two or more single-stranded antisense oligonucleotides linked at their 5′ ends” generally refers to a linkage between the 5′ terminal nucleotides of the oligonucleotides which may be directly via 5′, 3′ or 2′ hydroxyl groups, or indirectly, via a non-nucleotide linker. Such linkages may also be via a nucleoside, utilizing both 2′ and 3′ hydroxyl positions of the nucleoside. Such linkages may also utilize a functionalized sugar or nucleobase of a 5′terminal nucleotide.

The term “about” generally means that the exact number is not critical. Thus, oligonucleotides having one or two fewer nucleoside residues, or from one to several additional nucleoside residues are contemplated as equivalents of each of the embodiments described above.

The term “accessible” generally means when related to a compound according to the invention, that the relevant portion of the molecule is able to be recognized by the cellular components necessary to elicit an intended response to the compound.

The term “agonist” generally refers to a substance that binds to a receptor of a cell and induces a response. An agonist often mimics the action of a naturally occurring substance such as a ligand.

The term “antigen” generally refers to a substance that is recognized and selectively bound by an antibody or by a T cell antigen receptor. Antigens may include but are not limited to peptides, proteins, lipids, carbohydrates, nucleosides, nucleotides, nucleic acids, and combinations thereof. Antigens may be natural or synthetic and generally induce an immune response that is specific for that antigen.

“Antisense activity” means any detectable or measurable activity attributable to the hybridization of a gene silencing compound to its target nucleic acid. In certain embodiments, antisense activity is a decrease in the amount or expression of a target nucleic acid or protein encoded by such target nucleic acid.

“Gene silencing compound” means an oligomeric compound comprising two or more single stranded antisense oligonucleotides that are linked through their 5′-ends to allow the presence of two or more accessible 3′-ends. Gene silencing compounds are capable of undergoing hybridization to a target nucleic acid through hydrogen bonding.

“Antisense inhibition” means reduction of target nucleic acid levels or target protein levels in the presence of a gene silencing compound complementary to a target nucleic acid as compared to target nucleic acid levels or target protein levels in the absence of the gene silencing compound.

“Antisense oligonucleotide” means a single-stranded oligonucleotide having a nucleobase sequence that permits hybridization to a corresponding region or segment of a target nucleic acid.

The term “biologic instability” generally refers to a molecule's ability to be degraded and subsequently inactivated in vivo. For oligonucleotides, such degradation results from exonuclease activity and/or endonuclease activity, wherein exonuclease activity refers to cleaving nucleotides from the 3′ or 5′ end of an oligonucleotide, and endonuclease activity refers to cleaving phosphodiester bonds at positions other than at the ends of the oligonucleotide.

The term “carrier” generally encompasses any excipient, diluent, filler, salt, buffer, stabilizer, solubilizer, oil, lipid, lipid containing vesicle, microspheres, liposomal encapsulation, or other material for use in pharmaceutical formulations. It will be understood that the characteristics of the carrier, excipient or diluent will depend on the route of administration for a particular application. The preparation of pharmaceutically acceptable formulations containing these materials is described in, for example, Remington's Pharmaceutical Sciences, 18^{th} Edition, ed. A. Gennaro, Mack Publishing Co., Easton, Pa., 1990.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “complementary” is intended to mean the capacity for pairing between nucleobases of a first nucleic acid and a second nucleic acid.

“Contiguous nucleobases” means nucleobases immediately adjacent to each other.

The term “individual” or “subject” or “patient” generally refers to a mammal, such as a human.

“DUX4 nucleic acid” means any nucleic acid encoding DUX4. For example, in certain embodiments, a DUX4 nucleic acid includes a DNA sequence encoding DUX4, an RNA sequence transcribed from DNA encoding DUX4 (including genomic DNA comprising introns and exons), and an mRNA sequence encoding DUX4. “DUX4 mRNA” means an mRNA encoding a DUX4 protein.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Hybridization” means the annealing of complementary nucleic acid molecules. In certain embodiments, complementary nucleic acid molecules include an antisense compound and a target nucleic acid.

“Inhibiting DUX4 mRNA or protein expression” means reducing expression of DUX4 mRNA and/or protein levels in the presence of a gene silencing compound according to the invention as compared to expression of DUX4 mRNA and/or protein levels in the absence of a gene silencing compound according to the invention.

The term “linear synthesis” generally refers to a synthesis that starts at one end of an oligonucleotide and progresses linearly to the other end. Linear synthesis permits incorporation of either identical or non-identical (in terms of length, base composition and/or chemical modifications incorporated) monomeric units into an oligonucleotide.

The term “mammal” is expressly intended to include warm blooded, vertebrate animals, including, without limitation, humans, non-human primates, rats, mice, cats, dogs, horses, cattle, cows, pigs, sheep and rabbits.

The term “myopathic disease,” “myopathic disorder,” or “myopathy” means a disease of muscle tissue, where dysfunctional muscle fibers result in muscle weakness. Mypoathic diseases may be inherited, such as muscular dystrophies, or acquired.

The term “nucleoside” generally refers to compounds consisting of a sugar, usually ribose, deoxyribose, pentose, arabinose or hexose, and a purine or pyrimidine base.

The term “nucleotide” generally refers to a nucleoside comprising a phosphorous-containing group attached to the sugar.

The term “modified nucleoside” or “nucleotide derivative” generally is a nucleoside that includes a modified heterocyclic base, a modified sugar moiety, or any combination thereof. In some embodiments, the modified nucleoside or nucleotide derivative is a non-natural pyrimidine or purine nucleoside, as herein described. For purposes of the invention, a modified nucleoside or nucleotide derivative, a pyrimidine or purine analog or non-naturally occurring pyrimidine or purine can be used interchangeably and refers to a nucleoside that includes a non-naturally occurring base and/or non-naturally occurring sugar moiety. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “modified oligonucleotide” as used herein describes an oligonucleotide in which at least two of its nucleotides are covalently linked via a synthetic linkage, i.e., a linkage other than a phosphodiester linkage between the 5′ end of one nucleotide and the 3′ end of another nucleotide in which the 5′ nucleotide phosphate has been replaced with any number of chemical groups. The term “modified oligonucleotide” also encompasses 2′-0,4′-C-methylene-b-D-ribofuranosyl nucleic acids, arabinose nucleic acids, substituted arabinose nucleic acids, hexose nucleic acids, peptide nucleic acids, morpholino, and oligonucleotides having at least one nucleotide with a modified base and/or sugar, such as a 2′-O-substituted, a 5-methylcytosine and/or a 3′-O-substituted ribonucleotide.

The term “nucleic acid” encompasses a genomic region or an RNA molecule transcribed therefrom. In some embodiments, the nucleic acid is mRNA.

The term “linker” generally refers to any moiety that can be attached to an oligonucleotide by way of covalent or non-covalent bonding through a sugar, a base, or the backbone. The non-covalent linkage may be, without limitation, electrostatic interactions, hydrophobic interactions, π-stacking interactions, hydrogen bonding and combinations thereof. Non-limiting examples of such non-covalent linkage includes Watson-Crick base pairing, Hoogsteen base pairing, and base stacking. The linker can be used to attach two or more nucleosides or can be attached to the 5′ and/or 3′ terminal nucleotide in the oligonucleotide. Such linker can be either a non-nucleotide linker or a nucleoside linker.

The term “non-nucleotide linker” generally refers to a chemical moiety, other than a linkage directly between two nucleotides that can be attached to an oligonucleotide by way of covalent or non-covalent bonding. Preferably such non-nucleotide linker is from about 2 angstroms to about 200 angstroms in length, and may be either in a cis or trans orientation.

The term “internucleotide linkage” generally refer to a chemical linkage to join two nucleosides through their sugars (e.g. 3′-3′, 2′-3′, 2′-5′, 3′-5′, 5′-5′) consisting of a phosphorous atom and a charged, or neutral group (e.g., phosphodiester, phosphorothioate, phosphorodithioate or methylphosphonate) between adjacent nucleosides.

The term “oligonucleotide” refers to a polynucleoside formed from a plurality of linked nucleoside units, which may include, for example, deoxyribonucleotides or ribonucleotides, synthetic or natural nucleotides, phosphodiester or modified linkages, natural bases or modified bases natural sugars or modified sugars, or combinations of these components. The nucleoside units may be part of viruses, bacteria, cell debris or oligonucleotide-based compositions (for example, siRNA and microRNA). Such oligonucleotides can also be obtained from existing nucleic acid sources, including genomic or cDNA, but are preferably produced by synthetic methods. In certain embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain exemplary embodiments, these internucleoside linkages may be phosphodiester, phosphorothioate or phosphorodithioate linkages, or combinations thereof. In exemplary embodiments, the nucleotides of the synthetic oligonucleotides are linked by at least one phosphorothioate internucleotide linkage. The phosphorothioate linkages may be mixed Rp and Sp enantiomers, or they may be stereoregular or substantially stereoregular in either Rp or Sp form (see Iyer et al. (1995) Tetrahedron Asymmetry 6:1051-1054). In certain embodiments, one or more of the oligonucleotides within the antisense compositions of the invention contain one or more 2′-O,4′-C-methylene-b-D-ribofuranosyl nucleic acids, wherein the ribose is modified with a bond between the 2′ and 4′ carbons, which fixes the ribose in the 3′-endo structural conformation.

The term “peptide” generally refers to oligomers or polymers of amino acids that are of sufficient length and composition to affect a biological response, for example, antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization, and methylation.

The term “pharmaceutically acceptable” means a non-toxic material that does not interfere with the effectiveness of a compound according to the invention or the biological activity of a compound according to the invention.

The term “physiologically acceptable” refers to a non-toxic material that is compatible with a biological system such as a cell, cell culture, tissue, or organism. Preferably, the biological system is a living organism, such as a mammal, particularly a human.

The term “prophylactically effective amount” generally refers to an amount sufficient to prevent or reduce the development of an undesired biological effect.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Single-stranded oligonucleotide” means an oligonucleotide which is not hybridized to a complementary strand.

“Specifically hybridizable” refers to a gene silencing compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Targeting” or “targeted” means the process of design and selection of a gene silencing compound that will specifically hybridize to a target nucleic acid and induce a desired effect.

“Target nucleic acid,” “target RNA,” “target mRNA,” and “target RNA transcript” all refer to a nucleic acid capable of being targeted by gene silencing compounds.

“Target segment” means the sequence of nucleotides of a target nucleic acid to which a gene silencing compound is targeted. “5′ target site” refers to the 5′-most nucleotide of a target segment. “3′ target site” refers to the 3′-most nucleotide of a target segment.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

The term “gene expression” generally refers to process by which information from a gene is used in the synthesis of a functional gene product, which may be a protein. The process may involve transcription, RNA splicing, translation, and post-translational modification of a protein, and may include mRNA, preRNA, ribosomal RNA, and other templates for protein synthesis.

In certain embodiments provided are methods, compounds, and compositions for inhibiting DUX4 mRNA or protein expression. In certain embodiments the compounds are antisense oligonucleotides, double stranded or single-stranded siRNA compounds, or gene silencing compounds.

As used herein, gene silencing compounds according to the invention comprise two or more single-stranded antisense oligonucleotides linked at their 5′ ends, wherein the compounds have two or more accessible 3′ ends. The general structure of the oligonucleotide-based compounds of the invention may be described by the following formula I:

3′-Nn . . . N1N2N3N4-5′-L-5′-N8N7N6N5 . . . Nm-3′ (Formula I),

wherein L is a nucleotide linker or non-nucleotide linker; N1-N8, at each occurrence, is independently a nucleotide or nucleotide derivative; Nm and Nn, at each occurrence, are independently a nucleotide or nucleotide derivative; and wherein m and n are independently numbers from 0 to about 40.

In some embodiments, m and n are independently numbers from 15 to about 40.

The linkage at the 5′ ends of the component oligonucleotides is independent of the other oligonucleotide linkages and may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

In certain embodiments provided are gene silencing compounds targeted to a human DUX4 nucleic acid. In certain embodiments, the human DUX4 nucleic acid is the sequence set forth in GENBANK Accession No. NM_001306068.1 (variant 1, incorporated herein as SEQ ID NO: 12) or the sequence set forth in GENBANK Accession No. NM_001293798.1 (variant 2, incorporated herein as SEQ ID NO: 13).

| (SEQ ID NO: 12) | |
|---|---|
| 1 | atggccctcc cgacaccctc ggacagcacc ctccccgcgg aagcccgggg acgaggacgg |
| 61 | cgacggagac tcgtttggac cccgagccaa agcgaggccc tgcgagcctg ctttgagcgg |
| 121 | aacccgtacc cgggcatcgc caccagagaa cggctggccc aggccatcgg cattccggag |
| 181 | cccagggtcc agatttggtt tcagaatgag aggtcacgcc agctgaggca gcaccggcgg |
| 241 | gaatctcggc cctggcccgg gagacgcggc ccgccagaag gccggcgaaa gcggaccgcc |
| 301 | gtcaccggat cccagaccgc cctgctcctc cgagcctttg agaaggatcg ctttccaggc |
| 361 | atcgccgccc gggaggagct ggccagagag acgggcctcc cggagtccag gattcagatc |
| 421 | tggtttcaga atcgaagggc caggcacccg ggacagggtg gcagggcgcc cgcgcaggca |
| 481 | ggcggcctgt gcagcgcggc ccccggcggg ggtcaccctg ctccctcgtg ggtcgccttc |
| 541 | gcccacaccg gcgcgtgggg aacggggctt cccgcacccc acgtgccctg cgcgcctggg |
| 601 | gctctcccac agggggcttt cgtgagccag gcagcgaggg ccgcccccgc gctgcagccc |
| 661 | agccaggccg cgccggcaga ggggatctcc caacctgccc cggcgcgcgg ggatttcgcc |
| 721 | tacgccgccc cggctcctcc ggacggggcg ctctcccacc ctcaggctcc tcgctggcct |
| 781 | ccgcacccgg gcaaaagccg ggaggaccgg gacccgcagc gcgacggcct gccgggcccc |
| 841 | tgcgcggtgg cacagcctgg gcccgctcaa gcggggccgc agggccaagg ggtgcttgcg |
| 901 | ccacccacgt cccaggggag tccgtggtgg ggctggggcc ggggtcccca ggtcgccggg |
| 961 | gcggcgtggg aaccccaagc cggggcagct ccacctcccc agcccgcgcc cccggacgcc |
| 1021 | tccgcctccg cgcggcaggg gcagatgcaa ggcatcccgg cgccctccca ggcgctccag |
| 1081 | gagccggcgc cctggtctgc actcccctgc ggcctgctgc tggatgagct cctggcgagc |
| 1141 | ccggagtttc tgcagcaggc gcaacctctc ctagaaacgg aggccccggg ggagctggag |
| 1201 | gcctcggaag aggccgcctc gctggaagca cccctcagcg aggaagaata ccgggctctg |
| 1261 | ctggaggagc tttaggacgc ggggttggga cggggtcggg tggttcgggg cagggcggtg |
| 1321 | gcctctcttt cgcggggaac acctggctgg ctacggaggg gcgtgtctcc gccccgcccc |
| 1381 | ctccaccggg ctgaccggcc tgggattcct gccttctagg tctaggcccg gtgagagact |
| 1441 | ccacaccgcg gagaactgcc attctttcct gggcatcccg gggatcccag agccggccca |
| 1501 | ggtaccagca gacctgcgcg cagtgcgcac cccggctgac gtgcaaggga gctcgctggc |
| 1561 | ctctctgtgc ccttgttctt ccgtgaaatt ctggctgaat gtctcccccc accttccgac |
| 1621 | gctgtctagg caaacctgga ttagagttac atctcctgga tgattagttc agagatatat |
| 1681 | taaaatgccc cctccctgtg gatcctata |
| (SEQ ID NO: 13) | |
| 1 | atggccctcc cgacaccctc ggacagcacc ctccccgcgg aagcccgggg acgaggacgg |
| 61 | cgacggagac tcgtttggac cccgagccaa agcgaggccc tgcgagcctg ctttgagcgg |
| 121 | aacccgtacc cgggcatcgc caccagagaa cggctggccc aggccatcgg cattccggag |
| 181 | cccagggtcc agatttggtt tcagaatgag aggtcacgcc agctgaggca gcaccggcgg |
| 241 | gaatctcggc cctggcccgg gagacgcggc ccgccagaag gccggcgaaa gcggaccgcc |
| 301 | gtcaccggat cccagaccgc cctgctcctc cgagcctttg agaaggatcg ctttccaggc |
| 361 | atcgccgccc gggaggagct ggccagagag acgggcctcc cggagtccag gattcagatc |
| 421 | tggtttcaga atcgaagggc caggcacccg ggacagggtg gcagggcgcc cgcgcaggca |
| 481 | ggcggcctgt gcagcgcggc ccccggcggg ggtcaccctg ctccctcgtg ggtcgccttc |
| 541 | gcccacaccg gcgcgtgggg aacggggctt cccgcacccc acgtgccctg cgcgcctggg |
| 601 | gctctcccac agggggcttt cgtgagccag gcagcgaggg ccgcccccgc gctgcagccc |
| 661 | agccaggccg cgccggcaga ggggatctcc caacctgccc cggcgcgcgg ggatttcgcc |
| 721 | tacgccgccc cggctcctcc ggacggggcg ctctcccacc ctcaggctcc tcgctggcct |
| 781 | ccgcacccgg gcaaaagccg ggaggaccgg gacccgcagc gcgacggcct gccgggcccc |
| 841 | tgcgcggtgg cacagcctgg gcccgctcaa gcggggccgc agggccaagg ggtgcttgcg |
| 901 | ccacccacgt cccaggggag tccgtggtgg ggctggggcc ggggtcccca ggtcgccggg |
| 961 | gcggcgtggg aaccccaagc cggggcagct ccacctcccc agcccgcgcc cccggacgcc |
| 1021 | tccgcctccg cgcggcaggg gcagatgcaa ggcatcccgg cgccctccca ggcgctccag |
| 1081 | gagccggcgc cctggtctgc actcccctgc ggcctgctgc tggatgagct cctggcgagc |
| 1141 | ccggagtttc tgcagcaggc gcaacctctc ctagaaacgg aggccccggg ggagctggag |
| 1201 | gcctcggaag aggccgcctc gctggaagca cccctcagcg aggaagaata ccgggctctg |
| 1261 | ctggaggagc tttaggacgc ggggtctagg cccggtgaga gactccacac cgcggagaac |
| 1321 | tgccattctt tcctgggcat cccggggatc ccagagccgg cccaggtacc agcagacctg |
| 1381 | cgcgcagtgc gcaccccggc tgacgtgcaa gggagctcgc tggcctctct gtgcccttgt |
| 1441 | tcttccgtga aattctggct gaatgtctcc ccccaccttc cgacgctgtc taggcaaacc |
| 1501 | tggattagag ttacatctcc tggatgatta gttcagagat atattaaaat gccccctccc |
| 1561 | tgtggatcct atag |

Certain embodiments provide gene silencing compounds comprising two oligonucleotides each, independently, consisting of 12 to 30 nucleotides having a nucleobase sequence comprising a portion of at least 12 contiguous nucleobases complementary to an equal length portion of SEQ ID NO: 12 or SEQ ID NO: 13. Certain embodiments provide compounds comprising two oligonucleotides each, independently, consisting of 15 to 25 nucleotides having a nucleobase sequence comprising a portion of at least 12 contiguous nucleobases complementary to an equal length portion of SEQ ID NO: 12 or SEQ ID NO: 13. Certain embodiments provide compounds comprising a modified oligonucleotide consisting of 18 to 21 nucleotides having a nucleobase sequence comprising a portion of at least 12 contiguous nucleobases complementary to an equal length portion of SEQ ID NO: 12 or SEQ ID NO: 13. In certain embodiments, the two oligonucleotide of the gene silencing compound each, independently, comprise at least 9, at least 10, at least 11, at least 12, at least 13, at least 14, at least 15, at least 16, at least 17, at least 18, or at least 19 contiguous nucleobases complementary to an equal length portion of SEQ ID NO: 12 or SEQ ID NO: 13.

In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, at least 21, at least 22, or at least 23, contiguous nucleobases complementary to an equal length portion of SEQ ID NO: 12 or SEQ ID NO: 13.

Certain embodiments provide gene silencing compounds comprising two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, and is at least 80% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, and is at least 85% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, and is at least 90% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, and is at least 95% complimentary to SEQ ID NO: 12 or SEQ ID NO: 13.

Certain embodiments provide gene silencing compounds comprising two oligonucleotides each, independently, comprising at least 12 contiguous nucleobases of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29. In certain embodiments, the gene silencing compounds comprise two oligonucleotides each, independently, comprising at least 12 contiguous nucleobases of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 80% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 85% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 90% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 95% complimentary to SEQ ID NO: 12 or SEQ ID NO: 13.

Certain embodiments provide gene silencing compounds comprising two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 80% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 85% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 90% complimentary to SEQ ID NO: 12 or SEQ ID NO: 13. In certain embodiments, the gene silencing compounds comprise two oligonucleotides each, independently, comprising at least 12 contiguous nucleobases of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29, and is at least 95% complimentary to SEQ ID NO: 12 or SEQ ID NO: 13.

Certain embodiments provide gene silencing compounds comprising two oligonucleotides each, independently, consisting of 12 to 30 nucleotides having a nucleobase sequence comprising a portion of at least 12 contiguous nucleobases complementary to an equal length portion of SEQ ID NO: 12. Certain embodiments provide compounds comprising two oligonucleotides each, independently, consisting of 15 to 25 nucleotides having a nucleobase sequence comprising a portion of at least 12 contiguous nucleobases complementary to an equal length portion of SEQ ID NO: 12. Certain embodiments provide compounds comprising a modified oligonucleotide consisting of 18 to 21 nucleotides having a nucleobase sequence comprising a portion of at least 12 contiguous nucleobases complementary to an equal length portion of SEQ ID NO: 12. In certain embodiments, the two oligonucleotide of the gene silencing compound each, independently, comprise at least 9, at least 10, at least 11, at least 12, at least 13, at least 14, at least 15, at least 16, at least 17, at least 18, or at least 19 contiguous nucleobases complementary to an equal length portion of SEQ ID NO: 12.

In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, at least 21, at least 22, or at least 23 contiguous nucleobases complementary to an equal length portion of SEQ ID NO: 12. In certain embodiments, the two oligonucleotide of the gene silencing compound each, independently, comprise at least 19, at least 20, or at least 21 contiguous nucleobases complementary to an equal length portion of SEQ ID NO: 12.

Certain embodiments provide gene silencing compounds comprising two oligonucleotides each, independently, consisting of 12 to 30 nucleotides having a nucleobase sequence comprising a portion of at least 12 contiguous nucleobases complementary to an equal length portion of SEQ ID NO: 13. Certain embodiments provide compounds comprising two oligonucleotides each, independently, consisting of 15 to 25 nucleotides having a nucleobase sequence comprising a portion of at least 12 contiguous nucleobases complementary to an equal length portion of SEQ ID NO: 13. Certain embodiments provide compounds comprising a modified oligonucleotide consisting of 18 to 21 nucleotides having a nucleobase sequence comprising a portion of at least 12 contiguous nucleobases complementary to an equal length portion of SEQ ID NO: 13. In certain embodiments, the two oligonucleotide of the gene silencing compound each, independently, comprise at least 9, at least 10, at least 11, at least 12, at least 13, at least 14, at least 15, at least 16, at least 17, at least 18, or at least 19 contiguous nucleobases complementary to an equal length portion of SEQ ID NO: 13.

In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, at least 21, at least 22, or at least 23 contiguous nucleobases complementary to an equal length portion of SEQ ID NO: 13. In certain embodiments, the two oligonucleotide of the gene silencing compound each, independently, comprise at least at least 19, at least 20, or at least 21 contiguous nucleobases complementary to an equal length portion of SEQ ID NO: 13.

In certain embodiments, the nucleobase sequence of the oligonucleotides of the gene silencing compound are, independently, at least 90% complementary over its entire length to a nucleobase sequence of SEQ ID NO: 12 or SEQ ID NO: 13. In certain embodiments, the nucleobase sequence of the oligonucleotides of the gene silencing compound are, independently, at least 95% complementary over its entire length to a nucleobase sequence of SEQ ID NO: 12 or SEQ ID NO: 13. In certain embodiments, the oligonucleotides of the gene silencing compound are at least 99% complementary over its entire length to SEQ ID NO: 12 or SEQ ID NO: 13. In certain embodiments, the nucleobase sequence of the oligonucleotides of the gene silencing compound are 100% complementary over its entire length to a nucleobase sequence of SEQ ID NO: 12 or SEQ ID NO: 13.

In certain embodiments, the oligonucleotides of the gene silencing compound are, independently, 12 to 30 nucleotides in length. In other words, the oligonucleotides are from 12 to 30 linked nucleobases. In other embodiments, the oligonucleotides, independently, consist of 15 to 28, 18 to 24, 19 to 22, or 20 linked nucleobases. In certain such embodiments, the oligonucleotides, independently, consist of 8, 9, 10, 11, 12, 13, 14, 15, 16, 17, 18, 19, 20, 21, 22, 23, 24, 25, 26, 27, 28, 29, 30 linked nucleobases in length, or a range defined by any two of the above values.

In certain embodiments, the oligonucleotides of the gene silencing compound are, independently, 15 to 40 nucleotides in length. In other words, the oligonucleotides are from 15 to 40 linked nucleobases. In other embodiments, the oligonucleotides, independently, consist of 15 to 28, 18 to 24, 19 to 22, or 19 linked nucleobases. In certain such embodiments, the oligonucleotides, independently, consist of 8, 9, 10, 11, 12, 13, 14, 15, 16, 17, 18, 19, 20, 21, 22, 23, 24, 25, 26, 27, 28, 29, 30 linked nucleobases in length, or a range defined by any two of the above values.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for DUX4 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Certain embodiments provide a composition comprising a gene silencing compound as described herein, or a salt thereof, and a pharmaceutically acceptable carrier or diluent. Certain embodiments provide a composition comprising two or more gene silencing compounds as described herein, or a salt thereof, and a pharmaceutically acceptable carrier or diluent. The two or more gene silencing compounds can inhibit the mRNA or protein expression of the same target or can inhibit the mRNA or protein expression of different targets.

In certain embodiments, gene silencing compounds according to the invention comprise two identical or different sequences conjugated at their 5′-5′ ends via a phosphodiester, phosphorothioate or non-nucleoside linker. Gene silencing compounds according to the invention that comprise identical sequences are able to bind to a specific mRNA via Watson-Crick hydrogen bonding interactions and inhibit mRNA and protein expression. Gene silencing compounds according to the invention that comprise different sequences are able to bind to two or more different regions of one or more mRNA target and inhibit mRNA and protein expression. Such compounds are comprised of heteronucleotide sequences complementary to target mRNA and form stable duplex structures through Watson-Crick hydrogen bonding.

In certain embodiments, gene silencing compounds according to the invention are useful in treating and/or preventing diseases wherein inhibiting DUX4 expression would be beneficial. Gene silencing compounds according to the invention include, but are not limited to, antisense oligonucleotides comprising naturally occurring nucleotides, modified nucleotides, modified oligonucleotides and/or backbone modified oligonucleotides.

Representative non-nucleotide linkers are set forth in Table 1.

TABLE 1
Representative Non-Nucleotide Linkers

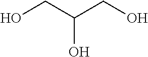

Glycerol (1,2,3-Propanetriol)

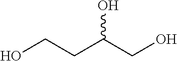

1,2,4-Butanetriol

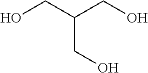

2-(hydroxymethyl)-1,3-propanediol

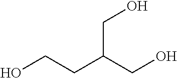

2-(hydroxymethyl)1,4-butanediol

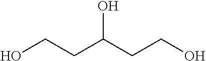

1,3,5-Pentanetriol

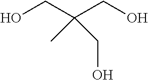

1,1,1-Tris(hydroxymethyl)ethane

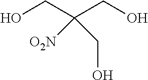

1,1,1-Tris(hydroxymethyl)nitromethane

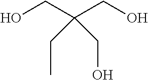

1,1,1-Tris(hydroxymethyl)propane

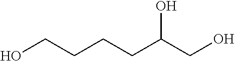

1,2,6-Hexanetriol

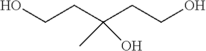

3-Methyl-1,3,5-pentanetriol

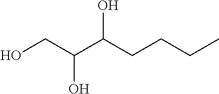

1,2,3-Heptanetriol

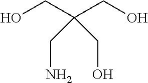

2-Amino-2-(hydroxymethyl)-1,3-propanediol

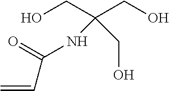

N-[Tris(hydroxymethyl)methyl]acrylamide

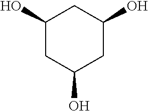

cis-1,3,5-Cyclohexanetriol

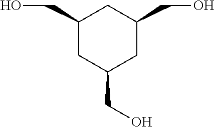 [SMILES: OC[C@H]1C[C@@H](CO)C[C@@H](CO)C1]

cis-1,3,5-Tri(hydroxymethyl)cyclohexane

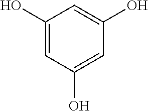

1,3,5,-Trihydroxyl-benzene

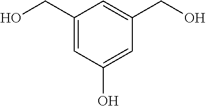 [SMILES: OCc1cc(O)cc(CO)c1]

3,5,-Di(hydroxymethyl)phenol

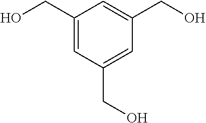 [SMILES: OCc1cc(CO)cc(CO)c1]

1,3,5,-Tri(hydroxymethyl)benzene

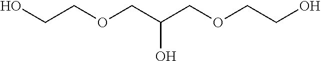 [SMILES: OCCOCC(O)COCCO]

1,3-Di(hydroxyethoxy)-2-hydroxyl-propane

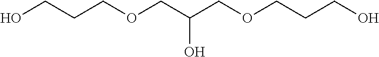 [SMILES: OCCCOCC(O)COCCCO]

1,3-Di(hydroxypropoxy)-2-hydroxyl-propane

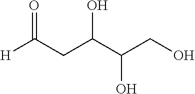

2-Deoxy-D-ribose

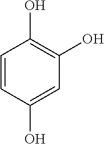

1,2,4,-Trihydroxyl-benzene

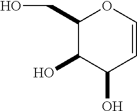

D-Galactoal

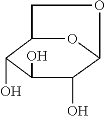 [SMILES: OC1C2COC(O2)C(O)C1O]

1,6-anhydro-β-D-Glucose

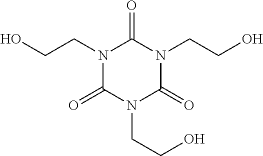 [SMILES: O=c1n(CCO)c(=O)n(CCO)c(=O)n1CCO]

1,3,5-Tris(2-hydroxyethyl)-Cyanuric acid

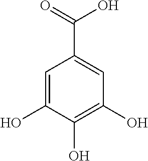 [SMILES: O=C(O)c1cc(O)c(O)c(O)c1]

Gallic acid

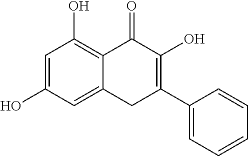 [SMILES: O=C1C(O)=C(c2ccccc2)Cc2cc(O)cc(O)c21]

3,5,7-Trihydroxyflavone

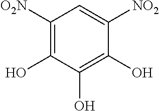 [SMILES: O=[N+]([O-])c1cc([N+](=O)[O-])c(O)c(O)c1O]

4,6-Nitropyrogallol

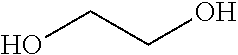

Ethylene glycol

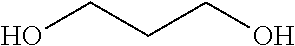

1,3-Propanediol

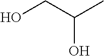

1,2-Propanediol

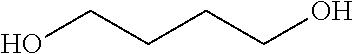

1,4-Butanediol

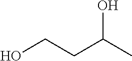

1,3-Butanediol

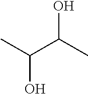

2,3-Butanediol

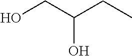

1,4-Butanediol

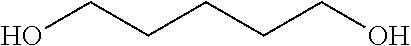

1,5-Pentanediol

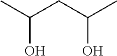

2,4-Pentanediol

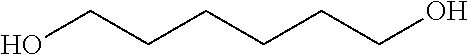

1,6-Hexanediol

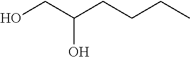

1,2-Hexanediol

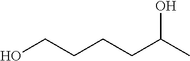

1,5-Hexanediol

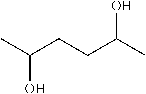

2,5-Hexanediol

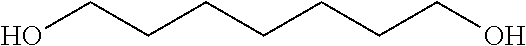

1,7-Heptanediol

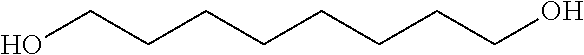

1,8-Octanediol

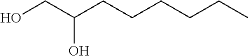

1,2-Octanediol

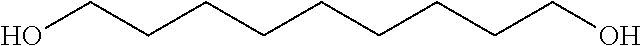 [SMILES: OCCCCCCCCCO]

1,9-Nonanediol

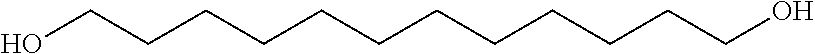 [SMILES: OCCCCCCCCCCCCO]

1,12-Dodecanediol

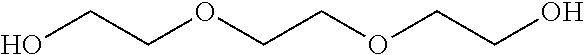

Triethylene glycol

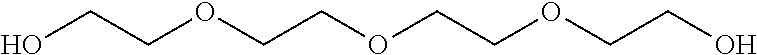 [SMILES: OCCOCCOCCOCCO]

Tetraethylene glycol

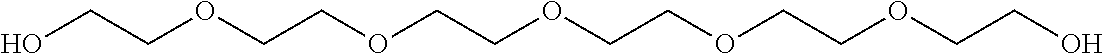 [SMILES: OCCOCCOCCOCCOCCOCCO]

Hexaethylene glycol

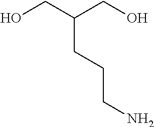

2-(1-Aminopropyl)-1,3-propanediol

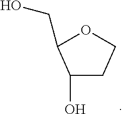

1,2-Dideoxyribose

In some embodiments, the small molecule linker is glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6 or from 2 to about 4.

In certain embodiments, the two or more oligonucleotides of the gene silencing compounds of the invention can be linked as shown in Table 2.

TABLE 2
Oligoribonucleotide Formulas II-V

| | |
|---|---|
| 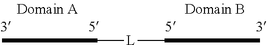 | Formula II |
| 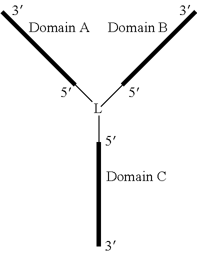 | Formula III |
| 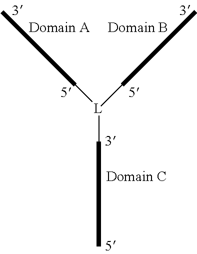 | Formula IV |
| 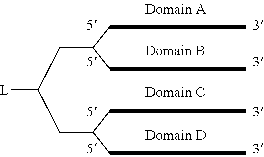 | Formula V |

In certain embodiments of Formulas II and/or V, L is a linker or a nucleotide linkage and Domain A and/or Domain B are antisense oligonucleotides that are designed to selectively hybridize to the same target RNA sequence or different target RNA sequences.

In certain embodiments of Formulas II, III, IV or V, L is a linker and Domain A and/or Domain B and/or Domain C and/or Domain D are antisense oligonucleotides that are designed to selectively hybridize to the same target RNA sequence or different target RNA sequences. For example, in one embodiment, Domain A and/or Domain B and/or Domain C of Formulas II and/or III are antisense oligonucleotides that are designed to selectively hybridize to the same target RNA sequence. In this embodiment, Domain A and/or Domain B and/or Domain C can be designed to hybridize to the same region on the target RNA sequence or to different regions of the same target RNA sequence.

In a further embodiment of this aspect of the invention, Domain A, Domain B, Domain C, and Domain D are independently RNA or DNA-based oligonucleotides. In certain aspects of this embodiment, the oligonucleotides comprise mixed backbone oligonucleotides.

In another embodiment, one or more of Domain A and/or Domain B and/or Domain C and/or Domain D is an antisense oligonucleotide that is designed to selectively hybridize to one target RNA sequence and one or more of the remaining Domain A and/or Domain B and/or Domain C and/or Domain D is an antisense oligonucleotide that is designed to selectively hybridized to a different target RNA sequence.

In another embodiment, one or more of Domain A and/or Domain B and/or Domain C and/or Domain D is an RNA-based oligonucleotide hybridized to a complimentary RNA-based oligonucleotide such that the domain comprises an siRNA molecule.

These gene silencing compounds of the invention can be prepared by the art recognized methods such as phosphoramidate or H-phosphonate chemistry which can be carried out manually or by an automated synthesizer. The synthetic antisense oligonucleotides of the invention may also be modified in a number of ways without compromising their ability to hybridize to mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate hydroxyl, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

The synthetic antisense oligonucleotides of the invention may comprise combinations of internucleotide linkages. For example, U.S. Pat. No. 5,149,797 describes traditional chimeric oligonucleotides having a phosphorothioate core region interposed between methylphosphonate or phosphoramidate flanking regions. Additionally, U.S. Pat. No. 5,652,356 discloses “inverted” chimeric oligonucleotides comprising one or more nonionic oligonucleotide region (e.g. alkylphosphonate and/or phosphoramidate and/or phosphotriester internucleoside linkage) flanked by one or more region of oligonucleotide phosphorothioate. Various synthetic antisense oligonucleotides with modified internucleotide linkages can be prepared according to standard methods. In certain embodiments, the phosphorothioate linkages may be mixed Rp and Sp enantiomers, or they may be made stereoregular or substantially stereoregular in either Rp or Sp form.

Other modifications of gene silencing compounds of the invention include those that are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as 2′-O,4′-C-methylene-b-D-ribofuranosyl, or arabinose instead of ribose, or a 3′, 5′-substituted oligonucleotide having a sugar which, at both its 3′ and 5′ positions, is attached to a chemical group other than a hydroxyl group (at its 3′ position) and other than a phosphate group (at its 5′ position).

Other examples of modifications to sugars of the oligonucleotide-based compounds of the invention include modifications to the 2′ position of the ribose moiety which include but are not limited to 2′-O-substituted with an —O-alkyl group containing 1-6 saturated or unsaturated carbon atoms, or with an —O-aryl, or —O-allyl group having 2-6 carbon atoms wherein such —O-alkyl, —O-aryl or —O-allyl group may be unsubstituted or may be substituted, for example with halo, hydroxyl, trifluoromethyl, cyano, nitro, acyl, acyloxy, alkoxy, carboxy, carbalkoxyl or amino groups. None of these substitutions are intended to exclude the presence of other residues having native 2′-hydroxyl group in the case of ribose or 2′ H— in the case of deoxyribose.

The gene silencing compounds according to the invention can comprise one or more ribonucleotides. For example, U.S. Pat. No. 5,652,355 discloses traditional hybrid oligonucleotides having regions of 2′-O-substituted ribonucleotides flanking a DNA core region. U.S. Pat. No. 5,652,356 discloses an “inverted” hybrid oligonucleotide that includes an oligonucleotide comprising a 2′-O-substituted (or 2′ OH, unsubstituted) RNA region which is in between two oligodeoxyribonucleotide regions, a structure that “inverted relative to the “traditional” hybrid oligonucleotides. Non-limiting examples of particularly useful oligonucleotides of the invention have 2′-O-alkylated ribonucleotides at their 3′, 5′, or 3′ and 5′ termini, with at least four, and in some exemplary embodiments five, contiguous nucleotides being so modified. Non-limiting examples of 2′-O-alkylated groups include 2′-O-methyl, 2′-O-ethyl, 2′-O-propyl, 2′-O-butyls and 2′-O-methoxy-ethyl.

The oligonucleotide-based compounds of the invention may conveniently be synthesized using an automated synthesizer and phosphoramidite approach further described in Example 1. In some embodiments, the oligonucleotide-based compounds of the invention are synthesized by a linear synthesis approach.

An alternative mode of synthesis is “parallel synthesis”, in which synthesis proceeds outward from a central linker moiety. A solid support attached linker can be used for parallel synthesis, as is described in U.S. Pat. No. 5,912,332. Alternatively, a universal solid support (such as phosphate attached controlled pore glass) support can be used.

Parallel synthesis of the oligonucleotide-based compounds of the invention has several advantages over linear synthesis: (1) parallel synthesis permits the incorporation of identical monomeric units; (2) unlike in linear synthesis, both (or all) the monomeric units are synthesized at the same time, thereby the number of synthetic steps and the time required for the synthesis is the same as that of a monomeric unit; and (3) the reduction in synthetic steps improves purity and yield of the final immune modulatory oligoribonucleotide product.

At the end of the synthesis by either linear synthesis or parallel synthesis protocols, the oligonucleotide-based compounds of the invention may conveniently be deprotected with concentrated ammonia solution or as recommended by the phosphoramidite supplier, if a modified nucleoside is incorporated. The product oligonucleotide-based compounds is preferably purified by reversed phase HPLC, detritylated, desalted and dialyzed.

In certain embodiments, the oligonucleotides of the gene silencing compound according to the invention are selected from the non-limiting list of the oligonucleotides shown in Table 3 below. The oligonucleotides shown in Table 3 have phosphorothioate (PS) linkages, but may also include phosphodiester linkages. Those skilled in the art will recognize, however, that other linkages, based on phosphodiester or non-phosphodiester moieties may be included.

TABLE 3

| Oligo #/ | Target | |
|---|---|---|
| SEQ ID NO | Site | Sequence (5′-3′) |
| 1 | 197 | GACCTCTCATTCTGAAACC |
| 2 | 402 | GATCTGAATCCTGGACTCC |
| 3 | 1117 | CCAGGAGCTCATCCAGCAG |
| 4 | 1258 | CCTAAAGCTCCTCCAGCAG |
| 5 | 1260 | GTCCTAAAGCTCCTCCAGC |
| 6 | 1319 | CCCAGGAAAGAAAGGCAGT |
| 7 | 1323 | GATGCCCAGGAAAGAAAGG |
| 8 | 1423 | AACAAGGGCACAGAGAGGC |
| 9 | 1498 | ATGTAACTCTAATCCAGGT |
| 10 | 1518 | CTGAACTAATCATCCAGGA |
| 11 | Control | ACTGTACTGAATCATAAGG |
| 14 | 63 | GGTCCAAACGAGTCTCCGT |
| 15 | 134 | CGTTCTCTGGTGGCGATGC |
| 16 | 202 | GGCGTGACCTCTCATTCTG |
| 17 | 621 | CCTCGCTGCCTGGCTCACG |
| 18 | 1162 | CCGTTTCTAGGAGAGGTTG |
| 19 | 1236 | CCGGTATTCTTCCTCGCTG |
| 20 | 1400 | GAGCTCCCTTGCACGTCAG |
| 21 | 1442 | AGCCAGAATTTCACGGAAG |
| 22 | 1479 | TTGCCTAGACAGCGTCGGA |
| 23 | 1506 | TCCAGGAGATGTAACTCTA |
| 24 | 1520 | CTCTGAACTAATCATCCAG |
| 25 | 1521 | TCTCTGAACTAATCATCCA |
| 26 | 1523 | TATCTCTGAACTAATCATC |
| 27 | 1525 | TATATCTCTGAACTAATCA |
| 28 | 1530 | TTTAATATATCTCTGAACT |
| 29 | 1535 | GGCATTTTAATATATCTCT |

Compound names for GSOs directed to DUX4 are based on the oligonucleotide target sites. For example, compound 5 comprising two copies of Oligo #5 (e.g., 3′-CGACCTCCTCGAAATCCTG-5′-X-5′-GTCCTAAAGCTCCTCCAGC-3′, wherein X represents a non-nucleotidic linker) will be referred to herein, for example, as “1260”, or “GSO 1260”, or “DUX4-1260” or “GSO DUX4-1260”. Additionally, a GSO comprising two different oligonucleotides such as Oligo #8 and Oligo #9 (e.g., 3′-CGGAGAGACACGGGAACAA-5′-X-5′-ATGTAACTCTAATCCAGGT-3′, wherein X represents a non-nucleotidic linker) will be referred to herein, for example, as “1423/1498”, “GSO 1423/1498”, or “DUX4-1423/1498” or “GSO DUX4-1423/1498”.

Certain embodiments provide gene silencing compounds comprising two oligonucleotides independently selected from the oligonucleotides listed in Table 3. In certain embodiments, the gene silencing compounds comprise two oligonucleotides each, independently, comprising the sequence of SEQ ID NO: 1, 2, 3, 4, 5, 6, 7, 8, 9, or 10, or combinations thereof. In certain embodiments, the oligonucleotides of the gene silencing compound are the same. In certain embodiments, the oligonucleotides of the gene silencing compounds are different.

In certain embodiments, the gene silencing compounds comprise two oligonucleotides each, independently, comprising the sequence of SEQ ID NO: 14, 15, 16, 17, 18, 19, 20, 21, 22, 23, 24, 25, 26, 27, 28, or 29, or combinations thereof. In certain embodiments, the oligonucleotides of the gene silencing compound are the same. In certain embodiments, the oligonucleotides of the gene silencing compounds are different.

In certain embodiments, the gene silencing compounds comprise two oligonucleotides each, independently, comprising the sequence of SEQ ID NO: 1, 2, 3, 4, 5, 6, 7, 8, 9, 10, 14, 15, 16, 17, 18, 19, 20, 21, 22, 23, 24, 25, 26, 27, 28, or 29 or combinations thereof. In certain embodiments, the oligonucleotides of the gene silencing compound are the same. In certain embodiments, the oligonucleotides of the gene silencing compounds are different.

The oligonucleotides of the gene silencing compounds are linked through their 5′-ends to allow the presence of two or more accessible 3′-ends. In certain embodiments, the oligonucleotides are linked through one or more of the non-nucleotide linkers listed in Table 1. In certain embodiments, a single linker listed in Table 1 is used to link the oligonucleotides of the gene silencing compounds. In certain embodiments, the linker is small molecule linker such as glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6 or from 2 to about 4.

In certain embodiments, the invention provides a composition comprising a gene silencing compound according to the invention and one or more vaccines, antigens, antibodies, cytotoxic agents, chemotherapeutic agents (both traditional chemotherapy and modern targeted therapies), kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

In certain embodiments, the invention provides a method for inhibiting DUX4 mRNA or protein expression, the method comprising contacting a cell with a gene silencing compound according to the invention. In certain embodiments, the cell can be contacted with two or more gene silencing compounds targeting different regions of DUX4.

In certain embodiments, the gene silencing compound targets anywhere within the region spanning from nucleobase 100 to 300, 150 to 250, 175 to 225, 180-220, 50 to 250, 100 to 250, 150 to 350, or 150 to 300 of SEQ ID NO: 12 or SEQ ID NO: 13. In certain embodiments, the gene silencing compound targets anywhere within the region spanning from nucleobase 300 to 500, 350 to 450, 375 to 425, 385-425, 250 to 450, 300 to 450, 350 to 550, or 350 to 500 of SEQ ID NO: 12 or SEQ ID NO: 13. In certain embodiments, the gene silencing compound targets anywhere within the region spanning from nucleobase 1150 to 1400, 1200 to 1350, 1225 to 1325, 1240-1300, 1050 to 1350, 1100 to 1350, 1200 to 1450, or 1200 to 1400 of SEQ ID NO: 12 or SEQ ID NO: 13. Also included within these embodiments are gene silencing compounds that target regions of SEQ ID NO: 12 or SEQ ID NO: 13 within the regions specified, i.e. sub-regions within the specified regions. By way of non-limiting example, a gene silencing compound of the invention may target anywhere within the region spanning from nucleobase 1200 to 1400, or it may target anywhere within the region spanning from 1253 to 1337, or 1302 to 1370, etc.

In certain embodiments, the gene silencing compound targets anywhere within the region spanning from nucleobase 1 to 100, 100 to 300, 150 to 250, 175 to 225, 180-220, 50 to 250, 100 to 250, 150 to 350, or 150 to 300 of SEQ ID NO: 12 or SEQ ID NO: 13. In certain embodiments, the gene silencing compound targets anywhere within the region spanning from nucleobase 300 to 500, 350 to 450, 375 to 425, 385-425, 250 to 450, 300 to 450, 350 to 550, 350 to 500, or 500 to 650 of SEQ ID NO: 12 or SEQ ID NO: 13. In certain embodiments, the gene silencing compound targets anywhere within the region spanning from nucleobase 1150 to 1400, 1200 to 1350, 1225 to 1325, 1240-1300, 1050 to 1350, 1100 to 1350, 1200 to 1450, 1200 to 1400, or 1400 to 1650 of SEQ ID NO: 12 or SEQ ID NO: 13. Also included within these embodiments are gene silencing compounds that target regions of SEQ ID NO: 12 or SEQ ID NO: 13 within the regions specified, i.e. sub-regions within the specified regions. By way of non-limiting example, a gene silencing compound of the invention may target anywhere within the region spanning from nucleobase 1200 to 1400, or it may target anywhere within the region spanning from 1253 to 1337, or 1302 to 1370, etc.

Certain embodiments further provide a method to reduce DUX4 mRNA or protein expression in an animal comprising administering to the animal a gene silencing compound or composition as described herein to reduce DUX4 mRNA or protein expression in the animal. In certain embodiments, the animal is a human. In certain embodiments, reducing DUX4 mRNA or protein expression prevents, treats, ameliorates, or slows progression of disease. In certain embodiments two or more gene silencing compounds targeting different regions of DUX4 can be administered.

In certain embodiments provided are methods for treating myopathic diseases or disorders comprising administering to the animal a gene silencing compound or composition as described herein to reduce DUX4 mRNA or protein expression in the animal. In certain embodiments the myophatic disease or disorder is a muscular dystrophy. In certain embodiments, the muscular dystrophy is FSHD. In certain embodiments, the animal is a human. In certain embodiments, the gene silencing compound or composition as described herein is administered intramuscularly. Thus, the inhibition of DUX4 mRNA or protein expression may provide a potentially useful strategy for patients with myopathic diseases or disorders. In certain embodiments two or more gene silencing compounds targeting different regions of DUX4 can be administered.

In certain embodiments provided are methods for treating diseases or disorders that would benefit from the reduced expression of a transcriptional activator of PITX1, or from modulation of PITX1, comprising administering to the animal a gene silencing compound or composition as described herein to reduce DUX4 mRNA or protein expression in the animal. In certain embodiments, the animal is a human. In certain embodiments two or more gene silencing compounds targeting different regions of DUX4 can be administered.

In certain embodiments provided are methods, compounds, and compositions for the treatment, prevention, or amelioration of diseases, disorders, and conditions associated with DUX4 in an individual in need thereof. Also contemplated are methods and compounds for the preparation of a medicament for the treatment, prevention, or amelioration of a disease, disorder, or condition associated with DUX4. In certain embodiments two or more gene silencing compounds targeting different regions of DUX4 can be administered.

DUX4 associated diseases, disorders, and conditions include myopathic diseases and any other disease, disorder or condition that would benefit from the modulation of DUX4 mRNA or protein expression. Myopathic diseases include muscular dystrophies, including FSHD.

In certain embodiments provided are DUX4 gene silencing compounds for use in treating, preventing, or ameliorating a DUX4 associated disease. In certain embodiments, DUX4 gene silencing compounds are capable of inhibiting the expression of DUX4 mRNA and/or DUX4 protein in a cell, tissue, or animal.

Certain embodiments provide methods comprising administering to an animal a gene silencing compounds as described herein. In certain embodiments two or more gene silencing compounds targeting different regions of DUX4 can be administered.

Also provided are methods and gene silencing compounds for the preparation of a medicament for the treatment, prevention, or amelioration of disease.

Certain embodiments provide the use of gene silencing compounds as described herein in the manufacture of a medicament for treating, ameliorating, or preventing disease.

Certain embodiments provide gene silencing compounds as described herein for use in treating, preventing, or ameliorating disease as described herein by combination therapy with an additional agent or therapy as described herein. Agents or therapies can be co-administered or administered concomitantly.

Certain embodiments provide the use of a gene silencing compound as described herein in the manufacture of a medicament for treating, preventing, or ameliorating disease as described herein by combination therapy with an additional agent or therapy as described herein. Agents or therapies can be co-administered or administered concomitantly.

Certain embodiments provide the use of a gene silencing compound as described herein in the manufacture of a medicament for treating, preventing, or ameliorating disease as described herein in a patient who is subsequently administered an additional agent or therapy as described herein.

In any of the methods according to the invention, the gene silencing compound according to the invention can variously act by producing direct gene expression modulation effects alone and/or in combination with any other agent useful for treating or preventing the disease or condition that does not diminish the gene expression modulation effect of the gene silencing compound according to the invention. In any of the methods according to the invention, the agent(s) useful for treating or preventing the disease or condition includes, but is not limited to, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modern targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the gene silencing compound according to the invention can be administered in combination with other compounds (for example lipids or liposomes) to enhance the specificity or magnitude of the gene expression modulation of the oligonucleotide-based compound according to the invention.

In any of the methods according to the invention, administration of gene silencing compounds according to the invention, alone or in combination with any other agent, can be by any suitable route, including, without limitation, intramuscular, parenteral, mucosal, oral, sublingual, intratumoral, transdermal, topical, inhalation, intrathecal, intranasal, aerosol, intraocular, intratracheal, intrarectal, vaginal, by gene gun, dermal patch or in eye drop or mouthwash form. In any of the methods according to the invention, administration of gene silencing compounds according to the invention, alone or in combination with any other agent, can be directly to a tissue or organ such as, but not limited to, the bladder, liver, lung, kidney or lung. In certain embodiments, administration of gene silencing compounds according to the invention, alone or in combination with any other agent, is by intramuscular administration. In certain embodiments, administration of gene silencing compounds according to the invention, alone or in combination with any other agent, is by mucosal administration. In certain embodiments, administration of gene silencing compounds according to the invention, alone or in combination with any other agent, is by oral administration. In certain embodiments, administration of gene silencing compounds according to the invention, alone or in combination with any other agent, is by intrarectal administration. In certain embodiments, administration of gene silencing compounds according to the invention, alone or in combination with any other agent, is by intrathecal administration.

Administration of the therapeutic compositions of gene silencing compounds according to the invention can be carried out using known procedures using an effective amount and for periods of time effective to reduce symptoms or surrogate markers of the disease. For example, an effective amount of a gene silencing compound according to the invention for treating a disease and/or disorder could be that amount necessary to alleviate or reduce the symptoms, or delay or ameliorate the disease and/or disorder. In the context of administering a composition that modulates gene expression, an effective amount of a gene silencing compound according to the invention is an amount sufficient to achieve the desired modulation as compared to the gene expression in the absence of the gene silencing compound according to the invention. The effective amount for any particular application can vary depending on such factors as the disease or condition being treated, the particular compound being administered, the size of the subject, or the severity of the disease or condition. One of ordinary skill in the art can empirically determine the effective amount of a particular compound without necessitating undue experimentation.

When administered systemically, the therapeutic composition is preferably administered at a sufficient dosage to attain a blood level of gene silencing compound according to the invention from about 0.0001 micromolar to about 10 micromolar. For localized administration, much lower concentrations than this may be effective, and much higher concentrations may be tolerated. Preferably, a total dosage of gene silencing compound according to the invention ranges from about 0.001 mg per patient per day to about 200 mg per kg body weight per day. In certain embodiments, the total dosage may be 0.08, 0.16, 0.32, 0.48, 0.32, 0.64, 1, 10 or 30 mg/kg body weight administered daily, twice weekly or weekly. It may be desirable to administer simultaneously, or sequentially a therapeutically effective amount of one or more of the therapeutic compositions of the invention to an individual as a single treatment episode.

The methods according to this aspect of the invention are useful for model studies of gene expression. The methods are also useful for the prophylactic or therapeutic treatment of human or animal disease. For example, the methods are useful for pediatric and veterinary inhibition of gene expression applications.

The examples below are intended to further illustrate certain preferred embodiments of the invention, and are not intended to limit the scope of the invention.

### Example 1

### Preparation of Oligonucleotide-Based Compounds

The oligonucleotide-based compounds of the invention were chemically synthesized using phosphoramidite chemistry on automated DNA/RNA synthesizer. TAC protected (Except U) 2′-O-TBDMS RNA monomers, A, G, C and U, were purchased from Sigma-Aldrich. 7-deaza-G, inosine and loxoribine monomers were purchased from ChemGenes Corporation. 0.25M 5-ethylthio-1H-tetrazole, PAC anhydride Cap A and Cap B were purchased from Glen Research. 3% trichloroacetic acid (TCA) in dichloromethane (DCM) and 5% 3H-1,2-Benzodithiole-3-one-1,1-dioxide (Beaucage reagent) were made in house.

Oligonucleotide-based compounds of the invention were synthesized at 1-2 μM scale using a standard DNA or RNA synthesis protocol.

### Cleavage and Base Deprotection

Oligonucleotide-based compounds of the invention were cleaved from solid support and the solution was further heated at 65° C. to removing protecting groups of exo cyclic-amines. The resulting solution was dried completely in a SpeedVac.

### IE HPLC Purification

Oligonucleotide-based compounds of the invention were purified by ion exchange HPLC.

Column: Dionex DNAPac 100 column (22×250)

Column Heater: ChromTech TL-105 HPLC column heater, temperature is set to 80° C.

Buffer A: 20 mM Tris-HCl, pH 7.0, 20% acetinitrile

Buffer B: 3.0 M NaCl, 20 mM Tris-HCl, pH 7.0, 20% acetonitrile

Flow rate: 10 ml/min

Gradient:
0-2 min: 0% B
2-11 min: 0% B to 35% B
11-41 min: 35% B to 90% B
41-45 min: 100% B

Crude solution of oligonucleotide-based compounds of the invention was injected into HPLC. Above gradient is performed and the fractions were collected. All fractions containing more than 90% desired product were mixed, and then the solution was concentrated to almost dry by RotoVap. RNAse-free water was added to make final volume of 10 ml.

### C-18 Reversed Phase Desalting

CC-18 Sep-Pak cartridge purchased from Waters was first conditioned with 10 ml of acetonitrile followed by 10 ml of 0.5 M sodium acetate. 10 ml of the solution of oligonucleotide-based compounds of the invention was loaded. 15 ml of water was then used to wash out the salt. The oligonucleotide-based compounds of the invention was eluted out by 1 ml of 50% acetonitrile in water.

The solution is placed in SpeedVac for 30 minutes. The remaining solution was filter through a 0.2 micro filter and then was lyophilized to dryness. The solid was then re-dissolved in water to make the desired concentration.

The final solution was stored below 0° C.

### Capillary Electrophoresis

Oligonucleotide-based compounds of the invention were analyzed by capillary electrophoresis according to the following conditions.
Instrument: Beckman 5010
Capillary: 62 cm ssDNA capillary
Sample preparation: 0.2 OD of oligonucleotide-based composition according to the invention was dissolved in 200 ul of RNAse-free water.
Injection: electro-kinetic injection at 5 KV for 5 seconds.
Running condition: 14 KV for 50 minutes at 30° C.

### Ion Exchange HPLC Analysis

Oligonucleotide-based compounds of the invention were analyzed by ion exchange HPLC according to the following conditions

Column: Dionex DNAPac guard column (22×250)

Column Heater: ChromTech TL-105 HPLC column heater, temperature is set to 80° C.

Buffer A: 100 mM Tris-HCl, pH 8.0, 20% acetinitrile

Buffer B: 2.0 M LiC1, 100 mM Tris-HCl, pH 8.0, 20% acetonitrile

Flow rate: 2 ml/min

Gradient:
0-2 min: 0% B
2-10 min: 0% B to 100% B
10-15 min: 100% B

### PAGE Analysis

0.3 OD of oligonucleotide-based compounds of the invention was loaded on 20% polyacrylamide gel and was running at constant power of 4 watts for approximately 5 hours. The gel was viewed under short wavelength UV light.

### In Vitro GSO Screening in Vector-Based Assays

In vitro screening of DUX4 gene silencing oligonucleotides (GSOs) was performed using a luciferase-reporter assay in Hepa1-6 cells. The luciferase reporter plasmid used was derived from psiCHECK™-2 vector (Promega, Madison, Wis.) by cloning the full-length transcript of human DUX4 downstream of the Renilla translational stop codon (Genewiz, Boston, Mass.). The psiCHECK™-2 Vector also contains a second reporter gene, firefly luciferase, which serves as an internal control to normalize transfection efficiency.

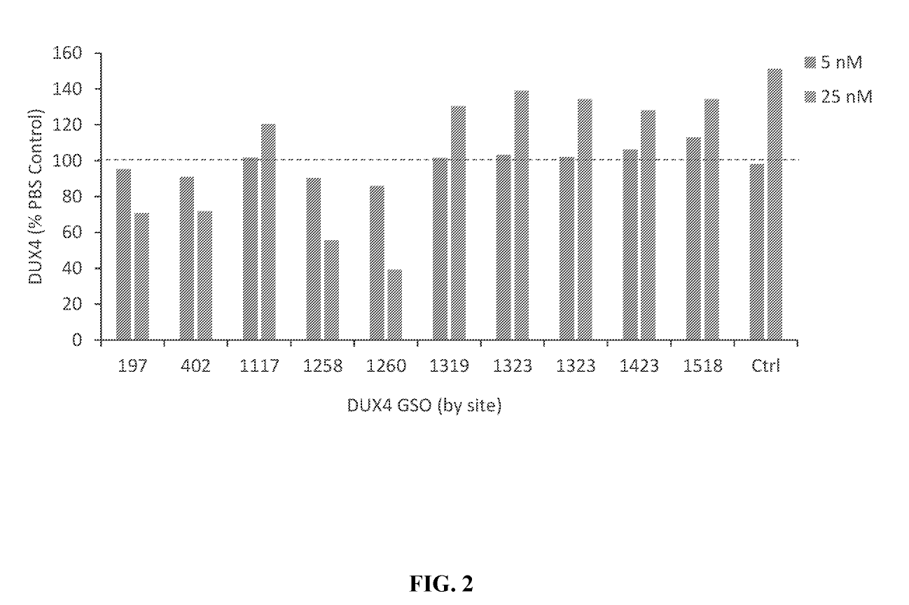

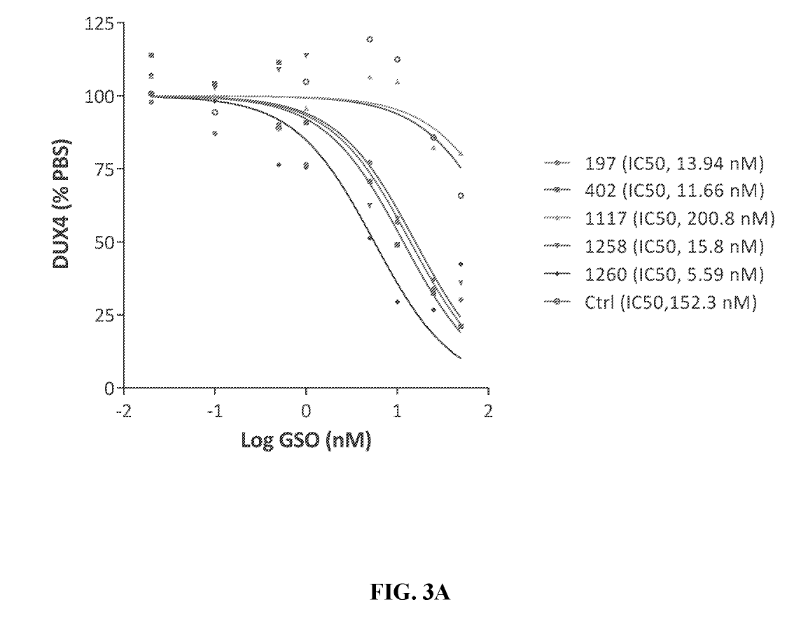

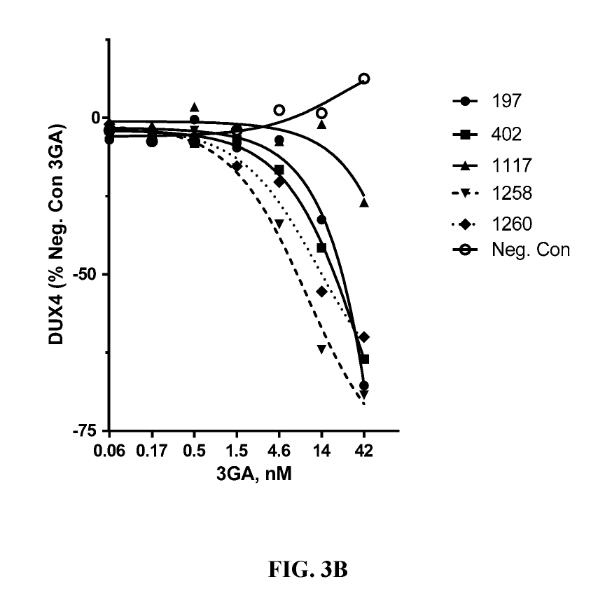

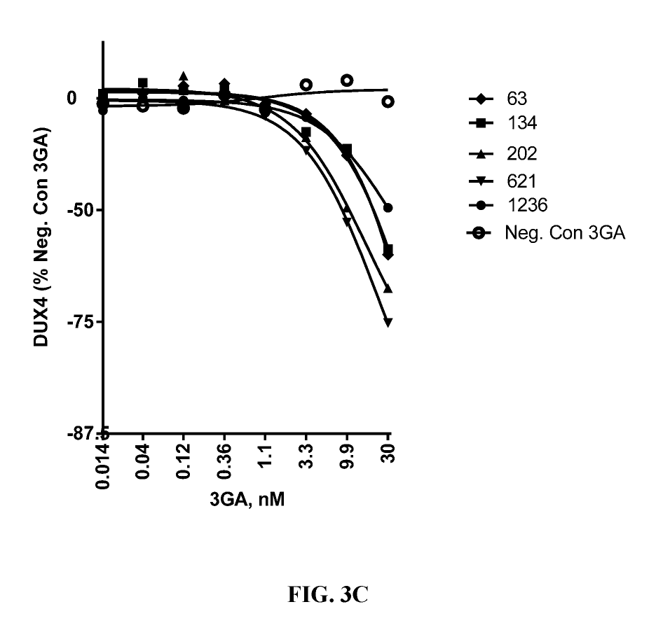

For primary screens, the GSOs shown in Table 4 were transfected at 5 and 25 nmol/l final concentration while for dose-response curve experiments, the GSOs were serially diluted starting at 50 nmol/l. Hepa1-6 cells (ATCC, Manassas, Va.) were maintained in DMEM (Dulbecco's Modified Eagle Medium) with 10% fetal bovine serum and 100 U/ml penicillin-streptomycin. For transfection, Hepa1-6 cells were seeded in 96-well white flat bottom plates at a density of 15,000 cells per well. The cells were reverse transfected with GSO and luciferase reporter plasmid together using Lipofectamine 2000 reagent (Life Technologies, Grand Island, N.Y.) and incubated at 37° C. overnight. The final concentrations of the reporter plasmid and Lipofectamine 2000 were 0.12 μg/ml and 3 respectively. Twenty-four hours post-transfection, reporter Renilla luciferase and control firefly luciferase activities were measured using Dual-Glo Luciferase Assay System (Promega). Normalized reporter activity was calculated by dividing Renilla activity by firefly activity of the same sample. The results are expressed as PBS control, and are provided in FIGS. 2 and 3.

TABLE 4
Exemplary gene silencing
compounds according to the invention.

| Oligo # | GSO sequence |
|---|---|
| 1 | 3′-CCAAAGTCTTACTCTCCAG-5′-X- |
| | 5′-GACCTCTCATTCTGAAACC-3′ |
| 2 | 3′-CCTCAGGTCCTAAGTCTAG-5′-X- |
| | 5′-GATCTGAATCCTGGACTCC-3′ |
| 3 | 3′-GACGACCTACTCGAGGACC-5′-X- |
| | 5′-CCAGGAGCTCATCCAGCAG-3′ |
| 4 | 3′-GACGACCTCCTCGAAATCC-5′-X- |
| | 5′-CCTAAAGCTCCTCCAGCAG-3′ |
| 5 | 3′-CGACCTCCTCGAAATCCTG-5′-X- |
| | 5′-GTCCTAAAGCTCCTCCAGC-3′ |
| 6 | 3′-TGACGGAAAGAAAGGACCC-5′-X- |
| | 5′-CCCAGGAAAGAAAGGCAGT-3′ |
| 7 | 3′-GGAAAGAAAGGACCCGTAG-5′-X- |
| | 5′-GATGCCCAGGAAAGAAAGG-3′ |
| 8 | 3′-CGGAGAGACACGGGAACAA-5′-X- |
| | 5′-AACAAGGGCACAGAGAGGC-3′ |
| 9 | 3′-TGGACCTAATCTCAATGTA-5′-X- |
| | 5′-ATGTAACTCTAATCCAGGT-3′ |
| 10 | 3′-AGGACCTACTAATCAAGTC-5′-X- |
| | 5′-CTGAACTAATCATCCAGGA-3′ |
| 11 | 3′-GGAATACTAAGTCATGTCA-5′-X- |
| (control) | 5′-ACTGTACTGAATCATAAGG-3′ |
| 14 | 3′-TGCCTCTGAGCAAACCTGG-5′-X- |
| | 5′-GGTCCAAACGAGTCTCCGT-3′ |
| 15 | 3′-CGTAGCGGTGGTCTCTTGC-5′-X- |
| | 5′-CGTTCTCTGGTGGCGATGC |
| 16 | 3′-GTCTTACTCTCCAGTGCGG-5′-X- |
| | 5′-GGCGTGACCTCTCATTCTG-3′ |
| 17 | 3′-GCACTCGGTCCGTCGCTCC-5′-X- |
| | 5′-CCTCGCTGCCTGGCTCACG-3′ |
| 19 | 3′-GTCGCTCCTTCTTATGGCC-5′-X- |
| | 5′-CCGGTATTCTTCCTCGCTG-3′ |

where X is glycerol.
The GSO sequence comprising Oligo # 11 was used as a control.

### EQUIVALENTS

Those skilled in the art will recognize, or be able to ascertain, using no more than routine experimentation, numerous equivalents to the specific substances and procedures described herein. For example, antisense oligonucleotides that overlap with the oligonucleotides may be used. Such equivalents are considered to be within the scope of this invention, and are covered by the following claims.

What is claimed is:

1. A synthetic oligonucleotide compound comprising two single stranded antisense oligonucleotides that are linked through their 5′-ends, each oligonucleotide, independently, comprise 12 to 30 nucleotides having a nucleobase sequence comprising a portion of at least 12 contiguous nucleobases complementary to an equal length portion of SEQ ID NO: 12 or SEQ ID NO: 13.

2. The compound according to claim 1, wherein the oligonucleotides are each, independently, between 15 and 40 nucleotides in length.

3. The compound according to claim 1, wherein the nucleobase sequence of each oligonucleotide is, independently, at least 90% complementary over its entire length to a nucleobase sequence of SEQ ID NO: 12 or SEQ ID NO: 13.

4. The compound according to claim 1, wherein each oligonucleotide, independently, comprises a portion of at least 12 contiguous nucleobases of SEQ ID NO: 5, 4, 2, 1, 16, 17, 19, 15, 14, 3, 6, 7, 8, 9, 10, 18, 20, 21, 22, 23, 24, 25, 26, 27, 28, or 29.

5. The compound according to claim 1, wherein each oligonucleotide, independently, comprises a portion of at least 12 contiguous nucleobases of SEQ ID NO: 5, 4, 2, 1, 16, 17, 19, 15, 14, 3, 6, 7, 8, 9, 10, 18, 20, 21, 22, 23, 24, 25, 26, 27, 28, or 29, and is at least 80% complimentary to its target site with SEQ ID NO: 12, SEQ ID NO: 13.

6. A composition comprising a compound according to claim 1 and a pharmaceutically acceptable carrier.

7. A synthetic oligonucleotide compound comprising two single stranded antisense oligonucleotides that are linked through their 5′-ends to allow the presence of two or more accessible 3′-ends, wherein the two oligonucleotides are the same and are selected from SEQ ID NOs: 5, 4, 2, 1, 16, 17, 19, 15, 14, 3, 6, 7, 8, 9, 10, 18, 20, 21, 22, 23, 24, 25, 26, 27, 28, or 29.

8. A composition comprising a compound according to claim 7 and a pharmaceutically acceptable carrier.

9. The composition according to claim 6, further one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

10. The composition according to claim 8, further one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

11. A method for inhibiting DUX4 mRNA or protein expression, the method comprising contacting a cell with at least one compound according to claim 1.

12. The method according to claim 11, wherein the cell is contacted with two or more compounds targeting different regions of DUX4.

13. A method for inhibiting DUX4 mRNA or protein expression, the method comprising contacting a cell with at least one compound according to claim 7.

14. The method according to claim 11, wherein the cell is contacted with two or more compounds targeting different regions of DUX4.

15. A method for inhibiting DUX4 mRNA or protein expression, the method comprising contacting a cell with at least one compound according to claim 8.

16. The method according to claim 15, wherein the cell is contacted with two or more compounds targeting different regions of DUX4.

17. A method for the treatment of a disease, disorder, or condition associated with DUX4 in an individual in need thereof, the method comprising administering a gene silencing compound according to claim 1.

18. A method for the treatment of a disease, disorder, or condition associated with DUX4 in an individual in need thereof, the method comprising administering a gene silencing compound according to claim 7.

19. A method for the treatment of a disease, disorder, or condition associated with DUX4 in an individual in need thereof, the method comprising administering a gene silencing compound according to claim 8.
